(19) United States
(12) Patent Application Publication
(10) Pub. No.: US 2015/0126533 A1
(43) Pub. Date: May 7, 2015

(54) ADMINISTRATION OF A RAF INHIBITOR AND A MEK INHIBITOR IN THE TREATMENT OF MELANOMA

(71) Applicant: TAKEDA PHARMACEUTICAL COMPANY LIMITED, Osaka-shi, Osaka (JP)

(72) Inventors: Takeo Arita, Kanagawa (JP); Shuntarou Tsuchiya, Kanagawa (JP)

(21) Appl. No.: 14/389,515

(22) PCT Filed: Mar. 29, 2013
(86) PCT No.: PCT/IB2013/052556

§ 371 Date: Sep. 30, 2014

Related U.S. Application Data
(60) Provisional application No. 61/618,006, filed on Mar. 30, 2012.

(51) Int. Cl. A61K 31/519; A61K 45/06; A61K 31/428

(52) U.S. Cl. CPC A61K 31/519; A61K 31/428; A61K 45/06
USPC 514/264.1

(57) ABSTRACT

Disclosed are methods for the treatment of non-BRAFV600E mutant melanoma in patients in need of such treatment. The methods comprise administering to such a patient a MEK inhibitor such as (R)-3-(2,3-dihydroxypropyl)-6-fluoro-5-((2-fluoro-4-iodophenyl)amino)-8-methylpyrido[2,3-d]pyrimidine-4,7(3H,8H)-dione (TAK-733) and a RAF inhibitor selected from N-{7-cyano-6-[4-fluoro-3-({[3-(trifluoromethyl)phenyl]acetyl}amino)phenoxy]-1,3-benzothiazol-2-yl}cyclopropanecarboxamide (TAK-632) and (R)-2-(1-(6-amino-5-chloropyrimidine-4-carboxamido)ethyl)-N-(5-chloro-4-(trifluoromethyl)pyridin-2-yl)thiazole-5-carboxamide (MLN2480). Also disclosed are medicaments for use in the treatment of such melanoma.

### FIELD

The present invention relates to the field of oncology and provides methods for treating melanoma.

### BACKGROUND

There is a continued need to find new therapeutic agents to treat human diseases. The MAPK/ERK kinases, such as MEK1 and MEK2, are especially attractive targets for the discovery of new therapeutics due to their important role in hyperproliferative disorders, diseases related to vasculogenesis or angiogenesis, T-cell mediated diseases where immune suppression would be of value, and other diseases. See, e.g., Q. Dong et al., Bioorg. & Med. Chem. Lett., 2011, 21, 1315-1319; and U.S. patent application Ser. No. 11/958,999 (U.S. Pat. No. 8,030,317). Compounds inhibiting RAF kinase have utility against cancers caused by mutation of growth factor receptor or excessive activation by ligand stimulation, or cancer caused by activation type mutation of Ras. See, e.g., U.S. patent application Ser. No. 12/628,697 (U.S. Pat. No. 8,143,258). Various RAF inhibitors have shown activity against melanomas with BRAFV600E mutation. See, e.g., P. I. Poulikakos and N. Rosen, Cancer Cell, 2011, 19, 11-15. However, even combinations of MEK and RAF inhibitors are not expected to demonstrate meaningful activity against melanoma outside of BRAFV600E mutant tumors. See, e.g., Poulikakos and Rosen.

The highest possible dose (MTD: maximum tolerated dose) is typically sought for agents for the treatment of cancer because the benefit of the treatment is believed to increase with dose. See, e.g., Y. Lin and W. J. Shih, Biostatistics, 2001, 2(2), 203-215. A synergistic combination of agents—that is, a combination of agents that is more effective than is expected from the effectiveness of its constituents—provides an opportunity to deliver even greater efficacy at the MTD, or to mitigate dose-related toxicity by delivering comparable efficacy with a lower dose. Accordingly, it is desirable to discover synergistic combinations of anti-cancer agents in order to treat cancer patients most effectively.

### SUMMARY

It has now been discovered that the administration of a MEK inhibitor and a particular RAF inhibitor provides a synergistic effect against non-BRAFV600E mutant melanoma.

In one aspect the invention relates to methods of treating non-BRAFV600E mutant melanoma comprising administering a MEK inhibitor and a RAF inhibitor selected from TAK-632 and MLN2480 to a subject in need of such treatment.

In one aspect, the invention relates to a kit comprising a medicament for use in treating non-BRAFV600E mutant melanoma in a subject in need of such treatment. The kit comprises a medicament comprising a MEK inhibitor, and instructions for administering the MEK inhibitor and the RAF inhibitor TAK-632 or MLN2480; or the kit comprises a medicament comprising the RAF inhibitor TAK-632 or MLN2480, and instructions for administering the RAF inhibitor and a MEK inhibitor. The kit can contain both a medicament comprising a MEK inhibitor and a medicament comprising the RAF inhibitor TAK-632 or MLN2480, and instructions for administering the MEK inhibitor and the RAF inhibitor.

In one aspect, the invention relates to a medicament for use in treating non-BRAFV600E mutant melanoma in a subject in need of such treatment. The medicament comprises a MEK inhibitor and a RAF inhibitor selected from TAK-632 and MLN2480.

### BRIEF DESCRIPTION OF THE DRAWINGS

FIG. 1 shows a fitted isobologram for TAK-632 (T-3109632) in combination with TAK-733 in the SK-Mel-30 cell line.

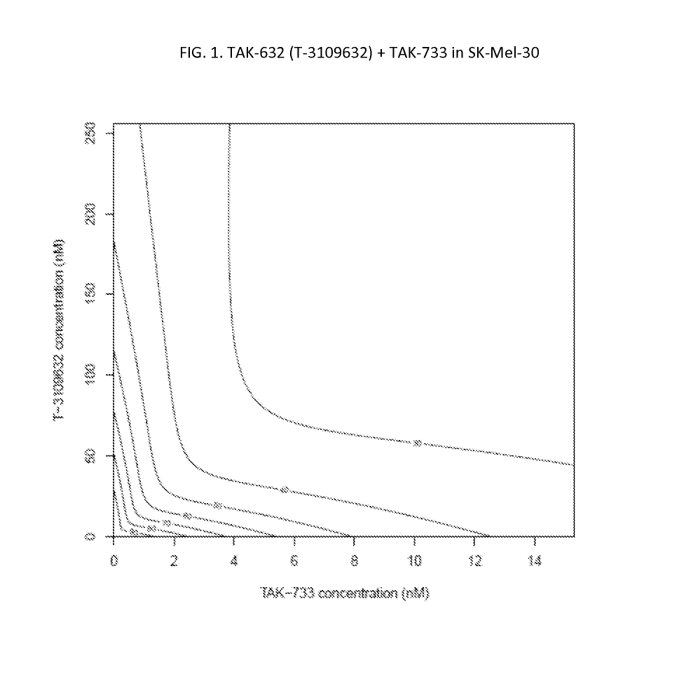

FIG. 2 shows a fitted isobologram for TAK-632 (T-3109632) in combination with TAK-733 in the SK-Mel-2 cell line.

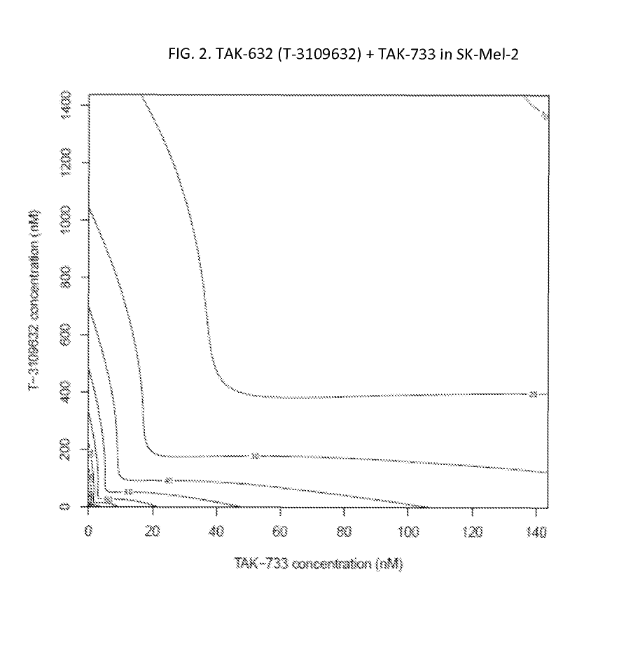

FIG. 3 shows a fitted isobologram for TAK-632 (T-3109632) in combination with TAK-733 in the IPC-298 cell line.

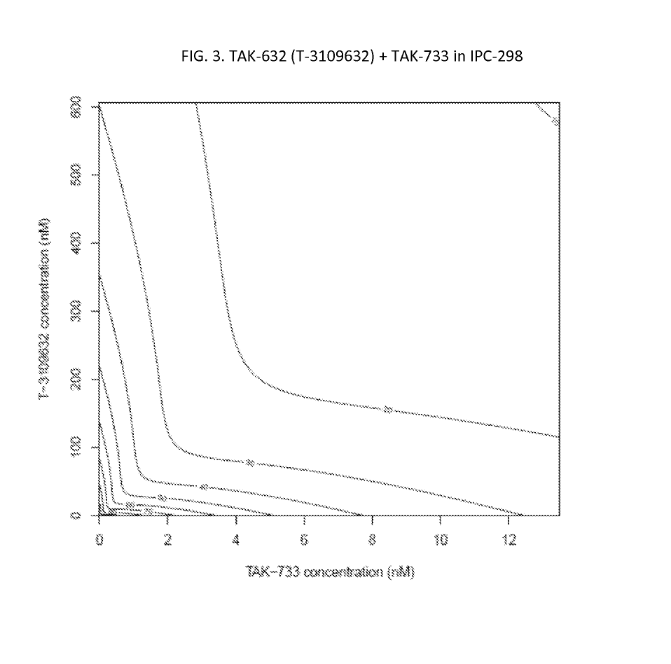

FIG. 4 shows a fitted isobologram for TAK-632 (T-3109632) in combination with TAK-733 in the MEL-JUSO cell line.

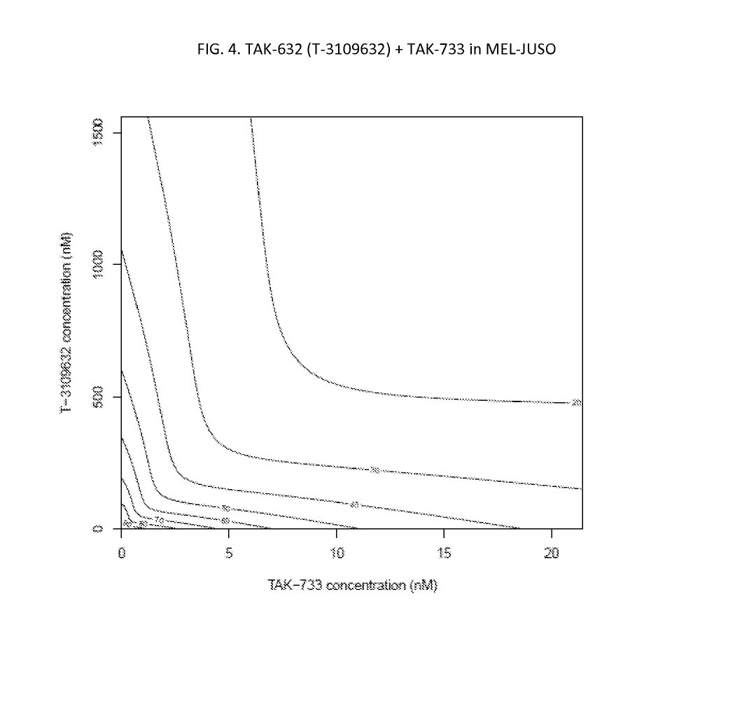

FIG. 5 shows a fitted isobologram for MLN2480 in combination with TAK-733 in the SK-Mel-30 cell line.

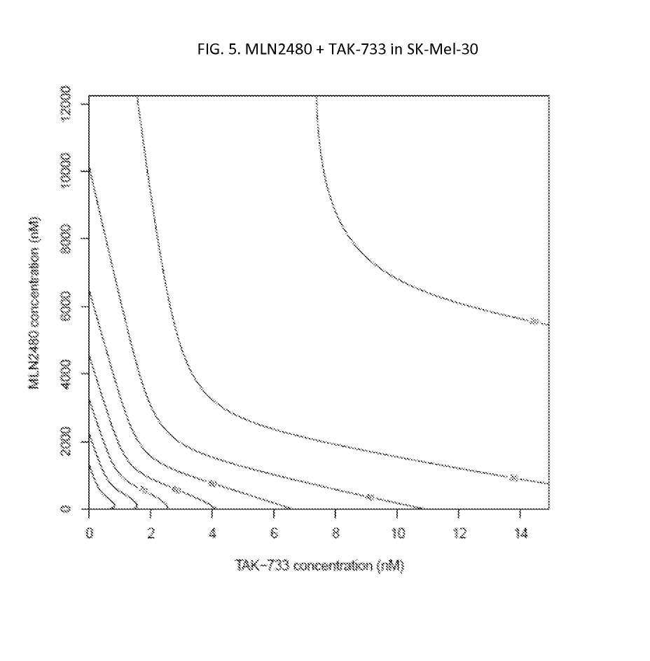

FIG. 6 shows a fitted isobologram for MLN2480 in combination with TAK-733 in the SK-Mel-2 cell line.

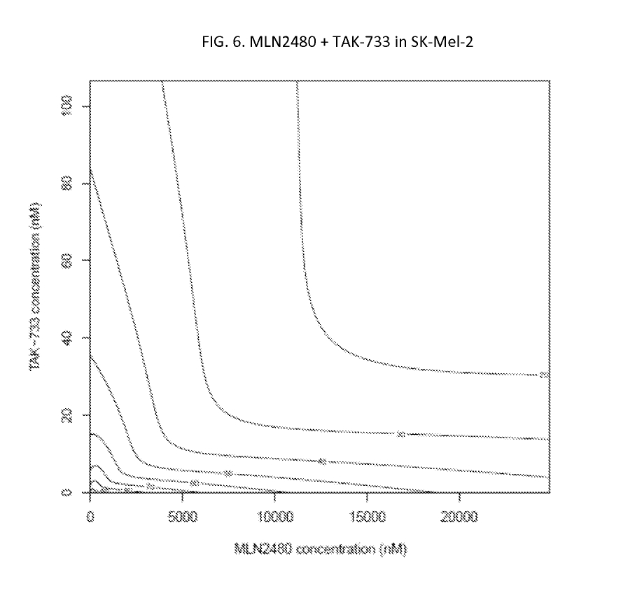

FIG. 7 shows a fitted isobologram for MLN2480 in combination with TAK-733 in the IPC-298 cell line.

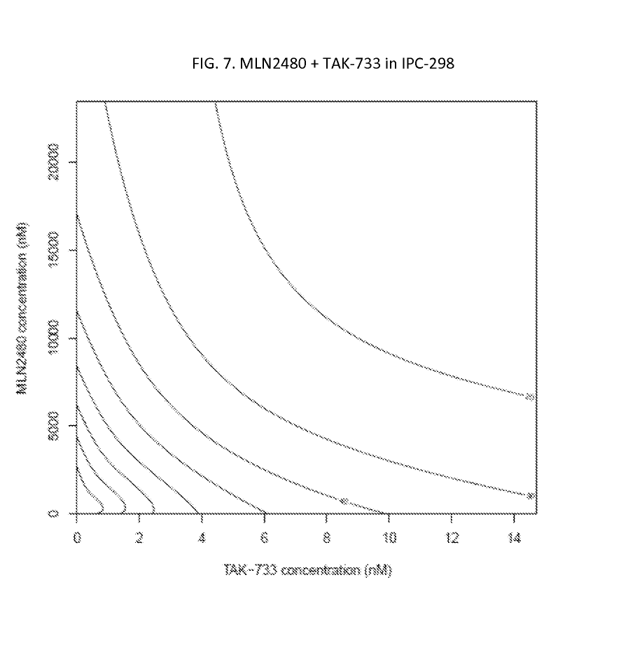

### DESCRIPTION

### Definitions and Abbreviations

As used herein, “therapeutically effective amount” means an amount of a therapeutic substance that is sufficient upon appropriate administration to a patient (a) to cause a detectable decrease in the severity of the disorder or disease state being treated; (b) to ameliorate or alleviate the patient's symptoms of the disease or disorder; or (c) to slow or prevent advancement of, or otherwise stabilize or prolong stabilization of, the disorder or disease state being treated (e.g., prevent additional tumor growth of a cancer).

When more than one therapeutic substance is being administered, the “therapeutically effective total amount” means that the sum of the individual amounts of each therapeutic substance meets the definition of “therapeutically effective amount” even if the individual amounts of any number of the individual therapeutic substances would not. For example, if 10 mg of A were not a therapeutically effective amount, and 20 mg of B were not a therapeutically effective amount, but the administration of 10 mg A+20 mg B resulted in at least one of the results enumerated for the definition of “therapeutically effective amount”, then the sum of 10 mg A+20 mg B would be considered a “therapeutically effective total amount”.

As used herein, “patient” means a human being diagnosed with, exhibiting symptoms of or otherwise believed to be afflicted with a disease, disorder or condition.

As used herein, the illustrative terms “include”, “such as”, “for example” and the like (and variations thereof, e.g., “includes” and “including”, “examples”), unless otherwise specified, are intended to be non-limiting. That is, unless explicitly stated otherwise, such terms are intended to imply “but not limited to”, e.g., “including” means including but not limited to.

### Therapeutic Substances—RAF Inhibitors.

The compound N-{7-cyano-6-[4-fluoro-3-({[3-(trifluoromethyl)phenyl]acetyl}amino)-phenoxy]-1,3-benzothiazol-2-yl}cyclopropanecarboxamide:

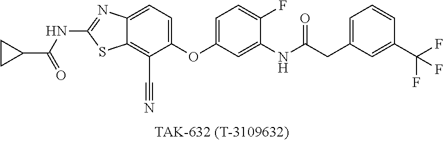 [SMILES: N#Cc1c(Oc2ccc(F)c(NC(=O)Cc3cccc(C(F)(F)F)c3)c2)ccc2nc(NC(=O)C3CC3)sc12]

also known as TAK-632 or T-3109632, is an inhibitor of Raf kinase. The compound (R)-2-(1-(6-amino-5-chloropyrimidine-4-carboxamido)ethyl)-N-(5-chloro-4-(trifluoromethyl)pyridin-2-yl)thiazole-5-carboxamide:

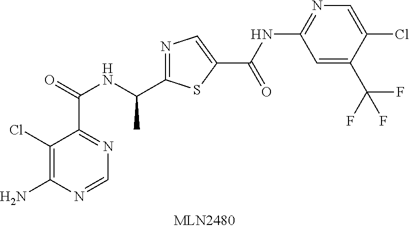 [SMILES: C[C@@H](NC(=O)c1ncnc(N)c1Cl)c1ncc(C(=O)Nc2cc(C(F)(F)F)c(Cl)cn2)s1]

also known as MLN2480, is also an inhibitor of Raf kinase. TAK-632, pharmaceutical compositions of thereof and processes for its synthesis have been described previously. See, e.g., U.S. patent application Ser. No. 12/628,697 (U.S. Pat. No. 8,143,258), which is hereby incorporated by reference herein in its entirety. MLN2480, pharmaceutical compositions of thereof and processes for its synthesis have been described previously. See, e.g., U.S. patent application Ser. No. 12/164,762 (Patent Appl. Publ. No. 2009/0036419), which is hereby incorporated by reference herein in its entirety. If there is any discrepancy between any of these documents and the present specification, the present specification controls.

### Therapeutic Substances—MEK Inhibitor.

The compound (R)-3-(2,3-dihydroxypropyl)-6-fluoro-5-((2-fluoro-4-iodophenyl)amino)-8-methylpyrido[2,3-d]pyrimidine-4,7(3H,8H)-dione:

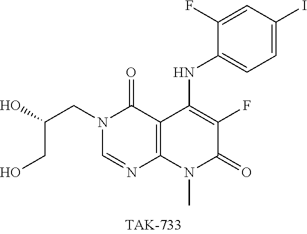 [SMILES: Cn1c(=O)c(F)c(Nc2ccc(I)cc2F)c2c(=O)n(C[C@@H](O)CO)cnc21]

also known as TAK-733, is a MEK inhibitor. See, e.g., Q. Dong et al., Bioorg. & Med. Chem. Lett., 2011, 21, 1315-1319, which is hereby incorporated by reference herein in its entirety. TAK-733, pharmaceutical compositions of thereof and processes for its synthesis have been described previously. See, e.g., U.S. patent application Ser. No. 11/958,999 (U.S. Pat. No. 8,030,317), which is hereby incorporated by reference herein in its entirety. If there is any discrepancy between any of these documents and the present specification, the present specification controls.

### Synergy.

It has now been discovered that the administration of a MEK inhibitor and a particular RAF inhibitor provides a synergistic effect against non-BRAFV600E mutant melanoma.

### In Vitro Combination Experiments.

As described in Example 1, cell viability assays were used to assess the combination effect in vitro of each of two RAF inhibitors, TAK-632 and MLN2480, with the MEK inhibitor TAK-733, in four BRAF wild-type NRAS mutant cell models of melanoma SK-Mel-30, SK-Mel-2 IPC-298 and MEL-JUSO. Table 1, below, lists the Combination Index (CI) and P-values for each determined combination, along with an assessment of synergy based on the CI value. As shown in Table 1, all of the tested combinations of TAK-632 and TAK-733 showed synergy, while 2 of the 3 tested combinations of MLN2480 and TAK-733 showed synergy.

TABLE 1
Combination Index values and Synergy Assessments.

| RAF | MEK | | Combination | P- | Con- |
|---|---|---|---|---|---|
| inhibitor | inhibitor | Cell line | Index | value | clusion |
| TAK-632 | TAK-733 | SK-Mel-30 | 0.38 | <0.001 | synergy |
| TAK-632 | TAK-733 | SK-Mel-2 | 0.24 | <0.001 | synergy |
| TAK-632 | TAK-733 | IPC-298 | 0.30 | <0.001 | synergy |
| TAK-632 | TAK-733 | MEL-JUSO | 0.38 | <0.001 | synergy |
| MLN2480 | TAK-733 | SK-Mel-30 | 0.54 | <0.001 | synergy |
| MLN2480 | TAK-733 | SK-Mel-2 | 0.37 | <0.001 | synergy |
| MLN2480 | TAK-733 | IPC-298 | 0.77 | <0.001 | additivity |

Table 3 (Example 2, below) lists the Combination Index (CI) values for each determined combination, along with an assessment of synergy based on the CI value. As shown in the table, the combination of TAK-632 and TAK-733 showed synergy in 4 of 4 tested NRAS mutant cell models of melanoma, 3 of which (SK-Mel-2, HMCB and GAK) are BRAF wild-type, and 1 of which (HMVII) is BRAFG469V mutant.

### Compound Administration

The RAF inhibitor can be administered in combination with the MEK inhibitor in a single dosage form or as a separate dosage form. When administered as a separate dosage form, the [symbol] the RAF inhibitor. As used herein, the administration in “combination” of RAF inhibitor and MEK inhibitor refers not only to simultaneous or sequential administration of the two agents, but also to the administration of both compounds during a single treatment cycle, as understood by one skilled in the art.

In various embodiments, the MEK inhibitor is TAK-733.

In various embodiments, the RAF inhibitor is TAK-632 or MLN2480. In various embodiments, the RAF inhibitor is TAK-632. In various embodiments, the RAF inhibitor is MLN2480.

### Therapeutic Substance; Pharmaceutical Compositions.

The therapeutic substance can be a pharmaceutically acceptable salt. In some embodiments, such salts are derived from inorganic or organic acids or bases. For reviews of suitable salts, see, e.g., Berge et al., J. Pharm. Sci., 1977, 66, 1-19 and Remington: The Science and Practice of Pharmacy, 20th Ed., A. Gennaro (ed.), Lippincott Williams & Wilkins (2000).

Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts, N-methyl-D-glucamine; and salts with amino acids such as arginine, lysine, and the like.

For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2-ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′-di(dehydroabietyl)-ethylenediamine), hydrobromide, hydrochloride, hydroxynaphthoate, iodide, isethionate (2-hydroxyethanesulfonate), lactate, lactobionate, malate, maleate, mandelate, mesylate (methanesulfonate), methylbromide, methylnitrate, methylsulfate, mucate, napsylate (2-naphthalenesulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′-dibenzylethylenediamine), chloroprocaine, choline, diethanolamine, ethylenediamine, meglumine (N-methylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis(salicylate), napadisylate (1,5-naphthalenedisulfonate), oxalate, pectinate, persulfate, phenylethylbarbiturate, picrate, propionate, thiocyanate, tosylate and undecanoate; organic cations benethamine (N-benzylphenethylamine), clemizole (1-p-chloro-benzyl-2-pyrrolildine-1′-ylmethylbenzimidazole), diethylamine, piperazine and tromethamine (tris(hydroxymethyl)aminomethane); and metallic cations barium and bismuth.

As used herein, “pharmaceutically acceptable carrier” refers to a material that is compatible with a recipient subject (a human) and is suitable for delivering an active agent to the target site without terminating the activity of the agent. The toxicity or adverse effects, if any, associated with the carrier preferably are commensurate with a reasonable risk/benefit ratio for the intended use of the active agent.

The pharmaceutical compositions for use in the methods of the invention can be manufactured by methods well known in the art such as conventional granulating, mixing, dissolving, encapsulating, lyophilizing, or emulsifying processes, among others. Compositions can be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations can contain stabilizers, pH modifiers, surfactants, solubilizing agents, bioavailability modifiers and combinations of these.

Pharmaceutically acceptable carriers that can be used in these compositions include ion exchangers, alumina, aluminum stearate, lecithin, serum proteins, such as human serum albumin, buffer substances such as phosphates or carbonates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

These pharmaceutical compositions are formulated for pharmaceutical administration to a human being. Such compositions can be administered orally, parenterally, by inhalation spray, topically, rectally, nasally, buccally, vaginally or via an implanted reservoir. The term “parenteral” as used herein includes subcutaneous, intravenous, intraperitoneal, intramuscular, intra-articular, intra-synovial, intrasternal, intrathecal, intrahepatic, intralesional and intracranial injection or infusion techniques. In some embodiments, the compositions are administered orally, intravenously or subcutaneously. In some embodiments, the compositions are administered orally. In some embodiments, the compositions are administered intravenously. These formulations can be designed to be short-acting, fast-releasing, or long-acting. Furthermore, the compositions can be administered in a local rather than systemic means, such as administration (e.g., by injection) at a tumor site.

Pharmaceutical formulations can be prepared as liquid suspensions or solutions using a liquid, such as an oil, water, an alcohol, and combinations of these. Solubilizing agents such as cyclodextrins can be included. Pharmaceutically suitable surfactants, suspending agents, or emulsifying agents, can be added for oral or parenteral administration. Suspensions can include oils, such as peanut oil, sesame oil, cottonseed oil, corn oil and olive oil. Suspension preparations can also contain esters of fatty acids such as ethyl oleate, isopropyl myristate, fatty acid glycerides and acetylated fatty acid glycerides. Suspension formulations can include alcohols, such as ethanol, isopropyl alcohol, hexadecyl alcohol, glycerol and propylene glycol; ethers, such as poly(ethyleneglycol); petroleum hydrocarbons such as mineral oil and petrolatum; and water.

Sterile injectable forms of these pharmaceutical compositions can be aqueous or oleaginous suspensions. These suspensions may be formulated according to techniques known in the art using suitable dispersing or wetting agents and suspending agents. The sterile injectable preparation can also be a sterile injectable solution or suspension in a non-toxic parenterally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms can also be used for the purposes of formulation. Compounds can be formulated for parenteral administration by injection such as by bolus injection or continuous infusion. A unit dosage form for injection can be in ampoules or in multi-dose containers.

These pharmaceutical compositions can be orally administered in any orally acceptable dosage form including capsules, tablets, aqueous suspensions or solutions. When aqueous suspensions are required for oral use, the active ingredient is combined with emulsifying and suspending agents. If desired, certain sweetening, flavoring or coloring agents can also be added. For oral administration in a capsule form, useful diluents include lactose and dried cornstarch. In the case of tablets for oral use, carriers that are commonly used include lactose and corn starch. Lubricating agents, such as magnesium stearate, are also typically added. Coatings may be used for a variety of purposes, e.g., to mask taste, to affect the site of dissolution or absorption, or to prolong drug action. Coatings can be applied to a tablet or to granulated particles for use in a capsule.

Alternatively, these pharmaceutical compositions can be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

These pharmaceutical compositions can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract may be effected in a rectal suppository formulation (see above) or in a suitable enema formulation. Topically-transdermal patches can also be used. For topical applications, the pharmaceutical compositions can be formulated in a suitable ointment containing the active component suspended or dissolved in one or more carriers. Carriers for topical administration of the compounds of this invention include mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active component(s) suspended or dissolved in one or more pharmaceutically acceptable carriers. Suitable carriers include mineral oil, sorbitan monostearate, polysorbate 60, cetyl esters wax, cetearyl alcohol, 2-octyldodecanol, benzyl alcohol and water.

For ophthalmic use, the pharmaceutical compositions can be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions can be formulated in an ointment such as petrolatum.

The pharmaceutical compositions can also be administered by nasal aerosol or inhalation. Such compositions are prepared according to techniques well known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

The methods of the invention are directed to treating diseases, disorders and conditions in which inhibition of RAF and MEK activity is detrimental to survival and/or expansion of diseased cells or tissue (e.g., cells are sensitive to such inhibition; inhibition of such activity disrupts disease mechanisms; reduction of such activity stabilizes protein which are inhibitors of disease mechanisms; reduction of such activity results in inhibition of proteins which are activators of disease mechanisms). The methods of the invention are particularly useful for the treatment of cancer. As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes solid tumors and bloodborne tumors. The term “cancer” encompasses diseases of skin, tissues, organs, bone, cartilage, blood, and vessels. The term “cancer” further encompasses primary and metastatic cancers.

In some embodiments, the cancer is non-BRAFV600E melanoma. In some embodiments, the cancer is NRAS mutant melanoma. In some embodiments, the cancer is BRAF wild-type melanoma. In some embodiments, the cancer is BRAF wild-type NRAS mutant melanoma.

In order that this invention be more fully understood, the following examples are set forth. These examples are illustrative only and are not intended to limit the scope of the invention in any way.

### EXAMPLES

### Example 1

### In Vitro Cell Viability Assays

The assay measures ATP concentration, a marker for cell viability. The CellTiter-Glo® Luminescent Cell Viability Assay (Promega, Madison, Wis.) is a homogenous method to determine the number of viable cells in culture based on quantitation of the ATP present. The experimental protocol uses Poly-D-lysine BioCoat™ Black/Clear 384 plates (Becton Dickinson, Franklin Lakes, N.J.). The SK-Mel-2 line was obtained from ATCC (American Type Culture Collection, Manassas, Va.), while the SK-Mel-30, IPC-298 and Mel-Juso lines were obtained from DSMZ (Deutsche Sammlung von Mikroorganismen and Zellkulturen GmbH, Brunswick, Germany). Each plate has a cell suspension from one of the lines added to the wells (25 μL/well), which is incubated (37° C., 6% CO₂) overnight or up to 24 hours. The appropriate inhibitors are dissolved in DMSO at varying concentrations and delivered into the wells using an Echo (Labcyte, Sunnyvale, Calif.) liquid handling system. The plates are incubated (37° C., 6% CO₂) for 72 hours. CellTiter-Glo® reagent, equilibrated at room temperature, is added (25 μL/well). After incubation for 10 min, the cell viability (luminescence) is measured using PHERAstar (BMG LABTECH, Orten berg, Germany).

### Statistical Analyses.

Normalization.

The viability data was normalized separately for each plate by scaling the data so that the median of the negative controls was 0 and the median of the positive controls was 100. More formally,

$${V}_{i} = 100 \frac{{U}_{i} - median ( {U}_{-} )}{median ( {U}_{+} ) - median ( {U}_{-} )}$$

where Vᵢ is the normalized viability of the i^{th} well, Uᵢ is the raw viability measurement, median(U₋) is the median of the negative controls, and median(U₊) is the median of the positive controls. After normalization, the controls were discarded.

Response Surface Model and Fitting.

A response surface model was used to describe the relationship between the normalized viability and the compound concentrations. For a given plate, let

C=(C_{A}/I₁)+(C_{B}/I₂)

x=(C_{A}/I₁)/C

Eₘₐₓ=E₁+E₂x+E₃x²+E₄x³

I=1+I₃x(1−x)

S=S₁+S₂x+S₃x²+S₄x³

V=100−Eₘₐₓ(1+(I/C)^{s})⁻¹+error

where E₁, E₂, E₃, E₄, I₁, I₂, I₃, S₁, S₂, S₃, and S₄ are parameters, C_{A} and C_{B} are the respective concentrations of compounds A and B, and V is the normalized viability measurement. It was assumed that the error values were independent and identically distributed normal random variables. This model is an extension of the Hill equation (A. V. Hill, J. Physiol., 1910, 40, iv-vii), which is commonly used to model the effect of a single compound. The data were fitted to this model using the maximum likelihood method with the statistical software program R R Development Core Team (2008) (R: A language and environment for statistical computing. R Foundation for Statistical Computing, Vienna, Austria. ISBN 3-900051-07-0, URL http://www.R-project.org).

Quality Checks.

Three types of quality checks were applied to the plates. First, it was checked that the variation of the positive controls and the mean of the negative controls were small. Next, it was checked that the new data agreed with data from previous single compound experiments. Finally, the residuals from the response surface fit were analyzed to ensure that the residual sum of squares was sufficiently small.

All of these quality checks were based on numerical thresholds to make pass/fail decisions, and the same thresholds were used for all of the plates in the experiment. If a plate failed any one of the quality checks, it was removed from the analysis.

Measuring Synergy.

The Combination Index (M. C. Berenbaum, J. Theor. Biol., 1985, 114, 413-431) was used as a measure of compound synergy. The Combination Index is computed based on an isobologram, which is a slice of the dose response surface with constant viability. For the present analysis, the 50% isobologram, which is the dose contour that has 50% viability, was used. The EC50_{A} and EC50_{B} are defined be the respective doses of the inhibitors (designated as compound A and B) alone that have a viability of 50%. For a point (D_{A}, D_{B}) along the 50% isobologram, the Combination Index is defined as (D_{A}/EC50_{A})+(D_{B}/EC50_{B}). Since the choice of (D_{A}, D_{B}) can be arbitrary, the constraint D_{A}/D_{B}=EC50_{A}/EC50_{B} was used. If the Combination Index is less than 0.7, it indicates that the 50% isobologram curves inward, and that the drug combination is synergistic. Conversely, if the Combination Index is greater than 1, the 50% isobologram curves outward, indicating antagonism.

### Example 2

### In Vitro Cell Growth Inhibition Assays

These assays show the effect of the combined use of TAK-632 and TAK-733 against human melanoma cell strains HMCB, HMV II, GAK and SK-MEL-2, which are reported to be NRAS mutations.

100 μl of cell suspension of human melanoma cells (HMCB, SK-MEL-2 (purchased from ATCC), GAK (purchased from HSRRB: Health Science Research Resources Bank) and HMV II (purchased from ECACC: European Collection of Cell Culture)) were inoculated in a 96-well plate (number of inoculated cells: HMV II 3000 cells/well; SK-MEL-2 2000 cells/well; HMCB 1000 cells/well; GAK 3000 cells/well), and cultured in a 5% carbon dioxide gas incubator at 37° C. The next day, solutions containing the tested compounds (TAK-632 and TAK-733) prepared such that the final concentrations were the combinations of concentrations shown in Table 2 were added in an amount of 100 μl to each well of the 96-well plate, and this was cultured for another 3 days. After culturing for 3 days, the solutions containing the tested compounds were removed from the wells of the 96-well plate, and washed using phosphate buffer solution (PBS). After washing, 50% trichloroacetate solution was added to each well so as to result in a final concentration of 10% (v/v), and this was left to stand overnight at 4° C. After leaving to stand overnight, 0.4% SRB (w/v) solution dissolved in 1% acetate was added in an amount of 50 μl/well, and the cell protein was fixed and stained (Skehan et al., Journal of the National Cancer Institute, Vol. 82, pp. 1107-1112, 1990). After staining, the wells of the plate were washed 3 times with 200 μl/well of 1% acetic acid solution, after which 100 μl of extract (10 mM tris buffer) was added to each well, and color extract was obtained. Absorbance (wavelength 550 nm) of the obtained color extract was measured. This study was performed for 3 wells for each combination of concentrations.

The measured growth inhibition rate was calculated from the formula below using the absorbance measured as described above. Measured growth inhibition rate (%)=(1−Absorbance of group to which tested compound was added/Absorbance of control group)×100

Additionally, the theoretical growth inhibition rate was calculated by the following method. Taking the protein mass of the control group to which no tested compound was added as 1, the protein mass of the compound-treated group was calculated, and the theoretical inhibition rate was determined from each protein mass based on the Bliss Independence Model (Bliss, C. I., Bacteriol. Rev. 20, 243-258 (1956)) and the Loewe Additive Model (Loewe, S., Arzneimittelforschung 3, 285-290 (1953)).

Conditions under which the growth inhibition rate measured when the two compounds TAK-632 and TAK-733 were combined was greater than the theoretical inhibition rate were seen for all cell strains, suggesting a synergistic growth inhibition effect by TAK-632 and TAK-733.

To judge the effect of combination, a study was performed using the Combination Index (CI) (Chou, T. C. & Talalay, P., J. Biol. Chem. 252, 6, 438-6442 (1977)). As a result, the CI value was 0.5 or below for all cells, and a synergistic growth inhibition effect of TAK-632 and TAK-733 was seen (Table 3). Additionally, when two-way analysis of covariance was performed on the measured values based on CI value, in all cell strains the growth inhibition effect when TAK-632 and TAK-733 were combined was significantly higher (p<0.01) than the growth inhibition effect when each of the compounds was used alone.

From these results, it was demonstrated that a significantly synergistic cell growth inhibition effect was obtained when the Pan-RAF inhibitor TAK-632 and the MEK inhibitor TAK-733 were used in combination in melanoma cell strains having a mutation in NRAS (Table 3).

TABLE 2

| | TAK-632 (nmol/L) | TAK-733 (nmol/L) |
|---|---|---|
| HMV II | 0, 3, 10, 30, 100, 300 | 0, 0.1, 0.3, 1, 3, 10, 30, 100, |
| | | 300, 1000 |
| SK-MEL-2 | 0, 10, 30, 100, 300, 1000 | 0, 0.1, 0.3, 1, 3, 10, 30, 100, |
| | | 300, 1000 |
| HMCB | 0, 30, 100, 300, 1000, 3000 | 0, 0.1, 0.3, 1, 3, 10, 30, 100, |
| | | 300, 1000 |
| GAK | 0, 30, 100, 300, 1000, 3000 | 0, 0.1, 0.3, 1, 3, 10, 30, 100, |
| | | 300, 1000 |

TABLE 3

| Cell line | Combination Index | Combination effect |
|---|---|---|
| HMVII | 0.404 | Synergistic |
| SK-MEL-2 | 0.313 | Synergistic |
| HMCB | 0.263 | Synergistic |
| GAK | 0.234 | Synergistic |

1-14. (canceled)

15. A method of treating melanoma comprising administering a therapeutically effective total amount of a MEK inhibitor and a RAF inhibitor to a patient in need of such treatment, wherein the RAF inhibitor is selected from N-{7-cyano-6-[4-fluoro-34 {[3-(trifluoromethyl)phenyl]acetyl}amino)-phenoxy]-1,3-benzothiazol-2-yl}cyclopropanecarboxamide or a pharmaceutically acceptable salt thereof and (R)-2-(1-(6-amino-5-chloropyrimidine-4-carboxamido)ethyl)-N-(5-chloro-4-(trifluoromethyl)pyridin-2-yl)thiazole-5-carboxamide or a pharmaceutically acceptable salt thereof and wherein the melanoma is non-BRAFV600E mutant.

16. The method according to claim 1, wherein the melanoma is BRAF wild-type.

17. The method according to claim 1, wherein the melanoma is NRAS mutant.

18. The method according to claim 1, wherein the melanoma is BRAF wild-type and NRAS mutant.

19. The method according to claim 1, wherein the MEK inhibitor is (R)-3-(2,3-dihydroxypropyl)-6-fluoro-5-((2-fluoro-4-iodophenyl)amino)-8-methylpyrido[2,3-d]-pyrimidine-4,7(3H,8H)-dione or a pharmaceutically acceptable salt thereof.

20. The method according to claim 19, wherein the RAF inhibitor is N-{7-cyano-6-[4-fluoro-3-({[3-(trifluoromethyl)phenyl]acetyl}amino)-phenoxy]-1,3-benzothiazol-2-yl}cyclopropanecarboxamide.

21. The method according to claim 19, wherein the RAF inhibitor is (R)-2-(1-(6-amino-5-chloropyrimidine-4-carboxamido)ethyl)-N-(5-chloro-4-(trifluoromethyl)pyridin-2-yl)thiazole-5-carboxamide.

22. A medicament comprising a RAF inhibitor and a MEK inhibitor, wherein the RAF inhibitor is selected from N-{7-cyano-6-[4-fluoro-3-({[3-(trifluoromethyl)-phenyl]acetyl}amino)-phenoxy]-1,3-benzothiazol-2-yl}cyclopropanecarboxamide or a pharmaceutically acceptable salt thereof and (R)-2-(1-(6-amino-5-chloropyrimidine-4-carboxamido)ethyl)-N-(5-chloro-4-(trifluoromethyl)pyridin-2-yl)thiazole-5-carboxamide or a pharmaceutically acceptable salt thereof.

23. The medicament of claim 22, wherein the MEK inhibitor is (R)-3-(2,3-dihydroxypropyl)-6-fluoro-5-((2-fluoro-4-iodophenyl)amino)-8-methylpyrido[2,3-d]-pyrimidine-4,7(3H,8H)-dione.

24. The medicament of claim 23, wherein the RAF inhibitor is N-{7-cyano-6-[4-fluoro-3-({[3-(trifluoromethyl)phenyl]acetyl}amino)-phenoxy]-1,3-benzothiazol-2-yl}cyclopropanecarboxamide.

25. The medicament of claim 23, wherein the RAF inhibitor is (R)-2-(1-(6-amino-5-chloropyrimidine-4-carboxamido)ethyl)-N-(5-chloro-4-(trifluoromethyl)pyridin-2-yl)thiazole-5-carboxamide.
